(19) United States
(12) Patent Application Publication
(10) Pub. No.: US 2025/0166789 A1
(43) Pub. Date: May 22, 2025

(54) METHOD AND APPARATUS FOR GENERATING ENHANCED TREATMENT PLANNING ANCILLARY DATA

(71) Applicant: GE Precision Healthcare LLC, Waukesha, WI (US)

(72) Inventors: Xueli Wang, Beijing (CN); Bingjie Zhao, Beijing (CN); Wenxian Jin, Beijing (CN)

(73) Assignee: GE Precision Healthcare LLC., Waukesha, WI (US)

(21) Appl. No.: 18/957,231

(22) Filed: Nov. 22, 2024

(30) Foreign Application Priority Data
Nov. 22, 2023 (CN) 202311566889.5

(51) Int. Cl. G16H 20/40; G06T 7/00; G06T 7/11

(52) U.S. Cl. CPC G16H 20/40; G06T 7/0016; G06T 7/11; G06T 2207/10072; G06T 2207/20104

(57) ABSTRACT

Provided are a method and an apparatus for generating enhanced treatment planning ancillary data. The example method includes acquiring a plurality of subsets of tomographic images of an object, where the plurality of subsets respectively correspond to different positions in a scanning axial direction, and where each of the plurality of subsets includes a plurality of tomographic images, at different time points of a specific scanning time period, obtained by scanning a movable portion of the object at a specific position in the scanning axial direction. The method further includes segmenting each of the plurality of tomographic images in one of the plurality of subsets to separately obtain a region of interest in each tomographic image and generating a region of interest contour of the one subset based on the regions of interest of the plurality of tomographic images in the one subset.

### CROSS-REFERENCE TO RELATED APPLICATIONS

This application claims priority to Chinese Application No. 202311566889.5, filed on Nov. 22, 2023, the disclosure of which is incorporated herein by reference in its entirety.

### TECHNICAL FIELD

The present disclosure relates to non-invasive diagnostic imaging and, more specifically, to methods and apparatus for generating enhanced treatment planning ancillary data.

### BACKGROUND

Radiotherapy has been used to treat tumors in human (and animal) tissues. In radiotherapy or radiosurgery, treatment planning is generally performed based on patients' medical images, and the treatment planning requires delineation of a target region and a normal critical organ in the medical images.

Computed tomography images are reference images for treatment planning, in particular dose planning, of a radiotherapy device. However, while a patient is in motion (e.g., breathing), it is challenging to accurately track various objects (e.g., a tumor, healthy tissue, or another aspect of the patients' anatomical structure). Therefore, it is necessary to learn a (four-dimensional) motion status of the objects over time.

A method of acquiring a patient's respiratory cycle has been developed to ensure that sufficient axial scanning is performed in a minimized duration at each axial position. When the patient's respiratory cycle is obtained, a doctor can observe a motion status of an object region within one respiratory cycle.

In an implementation of a respiratory gating technology known as Respiratory Gating for Scanners (RGSC), a patient wears an additional device. The device acquires images of the patient while CT simulation positioning is performed on the patient, and analyzes the correlation between the images and respiratory motion. In an implementation of another technology known as deviceless 4 dimension (D4D), a wire needs to be provided on a patient to perform helical pre-scanning, thereby predicting a scanning time required for subsequent axial scanning at one axial position. Axial scanning is then performed based on the predicted scanning time.

It is also necessary to learn motion pattern information of the objects, such as outer contour envelopes and other valuable information reflecting the motion of the objects, to allow a physician or technician to perform dose planning. Currently, physicians or technicians can only empirically estimate an outer contour envelope of a motion object to perform dose planning, and cannot accurately determine an area where irradiation of radiation needs to be applied.

### SUMMARY

The present disclosure is intended to overcome the above and/or other problems in the prior art, and can generate enhanced treatment planning ancillary data based on raw tomographic image data of an object to assist a physician in performing more accurate treatment planning.

According to a first aspect of the present disclosure, a computer implemented method is provided, including acquiring a plurality of subsets of tomographic images of an object, wherein the plurality of subsets respectively correspond to different positions in a scanning axial direction, and wherein each of the plurality of subsets comprises a plurality of tomographic images, at different time points of a specific scanning time period, obtained by scanning a movable portion of the object at a specific position in the scanning axial direction, segmenting each of the plurality of tomographic images in one of the plurality of subsets to separately obtain a region of interest in each tomographic image, and generating a region of interest contour of the one subset based on the regions of interest of the plurality of tomographic images in the one subset.

In an embodiment, the method may further include generating a probability distribution of pixels within a range of the region of interest contour of the one subset, wherein the probability distribution is a set of probabilities that respective pixels in the region of interest contour belong to regions of interest. In an embodiment, the probability distribution of the region of interest contour of the one subset is calculated using the one subset as a base. In an embodiment, the probability distribution of the region of interest contour of the one subset is calculated using the plurality of subsets as a base. In an embodiment, the region of interest contour is generated by superimposing contours of the regions of interest of all tomographic images in the same subset, such that pixels within the regions of interest of all the tomographic images in the same subset are located within the region of interest contour, and any pixel within the region of interest contour is located within the region of interest of at least one of all the tomographic images. In an embodiment, the region of interest is obtained by automatic segmentation via deep learning. In an embodiment, the method may further include defining, via a user input, a position range in which the region of interest is located. In an embodiment, the specific scanning time period of each subset exceeds one respiratory cycle of the object. In an embodiment, the specific scanning time period of at least one of the plurality of subsets is different from those of the other subsets. In an embodiment, the method may further include outputting the region of interest contour and the probability distribution together with the one subset to a treatment planning apparatus. In an embodiment, the method may further include displaying the region of interest contour in the tomographic images of the one subset, wherein the region of interest contour has transparency. In an embodiment, the region of interest contour of the one subset is presented in a manner corresponding to a probability distribution.

According to a second aspect of the present disclosure, a computer implemented method is provided. The method includes acquiring a plurality of subsets of tomographic images of an object, wherein the plurality of subsets respectively correspond to different positions in a scanning axial direction, and wherein each of the plurality of subsets comprises a plurality of tomographic images, at different time points of a specific scanning time period, obtained by scanning a movable portion of the object at a specific position in the scanning axial direction, segmenting each of the plurality of tomographic images in one of the plurality of subsets to separately obtain a region of interest in each tomographic image, and generating a probability distribution of pixels within a range of a region of interest contour of the one subset, wherein the probability distribution is a set of probabilities that respective pixels in the region of interest contour belong to regions of interest, and the region of interest contour is determined based on the regions of interest of all tomographic images in the one subset.

In an embodiment, the method may further include displaying the region of interest contour in the tomographic images of the one subset, wherein the region of interest contour has transparency. In an embodiment, the region of interest contour of the one subset is presented in a manner corresponding to the probability distribution.

According to a third aspect of the present disclosure, an image processing apparatus is provided, including a memory, storing a plurality of subsets of tomographic images of an object, and a processor, coupled to the memory and configured to perform the process described above.

According to a fourth aspect of the present disclosure, a non-transitory computer-readable storage medium is provided. The non-transitory computer-readable storage medium has instructions stored thereon, and when executed by a processor, the instructions cause the processor to perform the process described above.

According to a fifth aspect of the present disclosure, a treatment planning apparatus is provided, including an image acquisition unit, configured to acquire a plurality of subsets of tomographic images of an object and a region of interest contour corresponding to one of the plurality of subsets, wherein the region of interest contour is generated by superimposing contours of regions of interest obtained by segmentation of all tomographic images in the one subset, such that pixels within the regions of interest of all the tomographic images in the one subset are located within the region of interest contour, and any pixel within the region of interest contour is located within the region of interest of at least one of all the tomographic images; and a display, configured to present the region of interest contour corresponding to the one subset.

In an embodiment, the display is further configured to display the region of interest contour in the tomographic images of the one subset, wherein the region of interest contour has transparency. In an embodiment, the image acquisition unit is further configured to acquire a probability distribution of the region of interest contour, wherein the probability distribution is a set of probabilities that respective pixels in the region of interest contour belong to regions of interest; and the display is further configured to present the probability distribution of the region of interest contour corresponding to the one subset. In an embodiment, the display is further configured to present the region of interest contour of the one subset in a manner corresponding to the probability distribution.

### BRIEF DESCRIPTION OF THE DRAWINGS

The present disclosure can be better understood by describing exemplary embodiments of the present disclosure with reference to the drawings, in which:

FIG. 1 illustrates a schematic block diagram of an exemplary radiotherapy system according to an embodiment of the present disclosure;

FIG. 2 illustrates a schematic diagram of treatment planning incorporating enhanced ancillary data according to an embodiment of the present disclosure;

FIG. 3 illustrates a flowchart of an exemplary method for generating enhanced ancillary data according to an embodiment of the present disclosure;

FIG. 4 illustrates a schematic diagram of a 4D data set;

FIG. 5(A) to FIG. 5(C) illustrate exemplary tomographic images taken at different time points at the same scanning axial position;

FIG. 6 illustrates a schematic diagram of an exemplary region of interest contour according to an embodiment of the present disclosure;

FIG. 7 illustrates an exemplary generation method of a region of interest contour according to an embodiment of the present disclosure;

FIG. 8 illustrates a flowchart of another exemplary method for generating enhanced ancillary data according to an embodiment of the present disclosure;

FIG. 9 illustrates a schematic diagram of an exemplary probability distribution according to an embodiment of the present disclosure;

FIG. 10 illustrates a schematic diagram of an exemplary probability distribution of a plurality of subsets according to an embodiment of the present disclosure; and

FIG. 11 illustrates an exemplary block diagram of a computing device according to a techniques of the present disclosure.

In the accompanying drawings, similar components and/or features may have the same numerical reference signs. Further, components of the same type may be distinguished by letters following the reference sign, and the letters may be used for distinguishing between similar components and/or features. If only a first numerical reference sign is used in the specification, the description is applicable to any similar component and/or feature having the same first numerical reference sign irrespective of the subscript of the letter.

### DETAILED DESCRIPTION

Specific implementations of the present invention will be described below. It should be noted that in the specific description of these implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

References in the specification to “an embodiment,” “embodiment,” “exemplary embodiment,” and so on indicate that the embodiment described may include a specific feature, structure, or characteristic, but the specific feature, structure, or characteristic is not necessarily included in every embodiment. Besides, such phrases do not necessarily refer to the same embodiment. Further, when a specific feature, structure, or characteristic is described in connection with an embodiment, it is believed that affecting such feature, structure, or characteristic in connection with other embodiments (whether or not explicitly described) is within the knowledge of those skilled in the art.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

Unless defined otherwise, technical terms or scientific terms used in the claims and description should have the usual meanings that are understood by those of ordinary skill in the technical field to which the present invention belongs. The terms “include” or “comprise” and similar words indicate that an element or object preceding the terms “include” or “comprise” encompasses elements or objects and equivalent elements thereof listed after the terms “include” or “comprise”, and do not exclude other elements or objects.

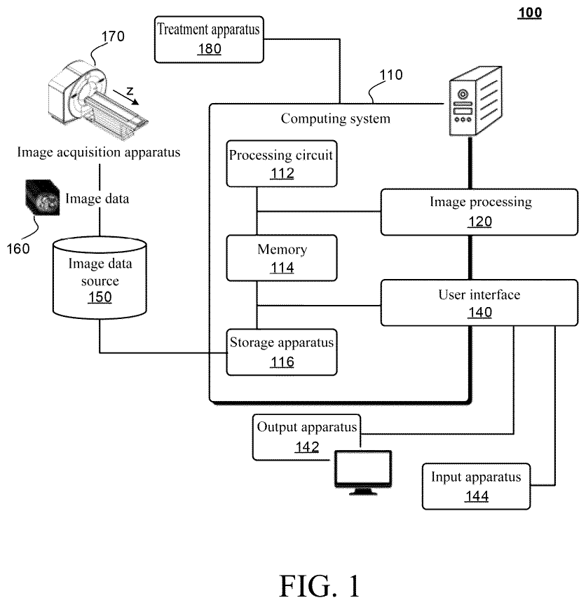

The implementations of the present disclosure will be described below by way of example with reference to FIG. 1 to FIG. 11, wherein the following description relates to various examples of imaging systems. Specifically, provided are a method and an apparatus for generating enhanced treatment planning ancillary data. The enhanced treatment planning ancillary data, as mentioned herein, is generated based on a raw tomographic image data set of an object, and can reflect a motion status of a movable region of interest. Thus, the enhanced treatment planning ancillary data of the present disclosure facilitates execution of treatment planning for radiotherapy. To better understand the present disclosure, an exemplary implementation of a radiotherapy system is first described below.

Image-guided radiotherapy (IGRT) is a technique that make use of imaging on a patient in a treatment position immediately prior to radiation. This enables more accurate targeting on an anatomical structure, such as an organ, a tumor, or an organ at risk. If the patient is expected to move during treatment, such as respiration-induced motion (which produces quasi-periodic motion of a lung tumor) or bladder filling leading to a drift in prostate position, additional margins may be arranged around a target to encompass desired patient motion. However, these relatively large margins may lead to increased side effects at the expense of high doses to surrounding normal tissues. Therefore, it is desirable to provide margins as accurate as possible.

The IGRT may use computed tomography (CT) imaging, cone-beam CT (CBCT), magnetic resonance (MR) imaging, positron emission tomography (PET) imaging, or the like prior to radiotherapy to obtain a 3D or 4D data set of a patient. For example, a CBCT-enabled linac (linear accelerator) may consist of a kV source/detector that is fixed to a gantry at a 90-degree angle to a radiation beam, or an MR linear accelerator apparatus may consist of a linear accelerator that is directly integrated with an MR scanner.

The 3D or 4D data set of the patient obtained using CT, CBCT, MR, or the like can be used for performing dose planning of radiotherapy by a physician. A patient model in the 4D data set may include a patient state that varies with a single parameter (e.g., a phase in a respiratory cycle). In other words, the 4D data set contains a plurality of subsets of tomographic images of the patient. Each subset corresponds to one position in a scanning axial direction (direction z in FIG. 1) and contains tomographic images at different time points at the position. Thus, one subset can reflect a local motion status of the patient at the axial position.

In the present disclosure, a 4D data set of a patient acquired by an image acquisition apparatus of CT, CBCT, MR, or the like is processed, and an outer contour envelope of an object (such as a tumor, an organ, or an organ at risk) is generated for at least one subset in the 4D data set to help a physician perform more accurate treatment planning, such as irradiation region setting and dose planning. The outer contour envelope is generated based on a region of interest in a tomographic image in the subset, and reflects a motion status of the region of interest over time. Therefore, more accurate treatment planning can be implemented based on the generated outer contour envelope.

FIG. 1 illustrates a schematic block diagram of an exemplary radiotherapy system 100 according to an embodiment of the present disclosure. Enhanced treatment planning ancillary data can be generated based on data in a 4D data set of a patient. The radiotherapy system 100 includes an image processing computing system 110 that hosts image processing logic 120. The image processing computing system 110 may be connected to a network (not shown), and such a network can be connected to the Internet. For example, the network may connect the image processing computing system 110 with one or more medical information sources (e.g., a radiation information system (RIS), a medical record system (e.g., an electronic medical record (EMR)/electronic health record (EHR) system), or an oncology information system (OIS)), one or more image data sources 150, an image acquisition apparatus 170, and a treatment apparatus 180 (e.g., a radiotherapy apparatus). As an example, the image processing computing system 110 may be configured to: perform an image processing operation, which is part of operations for generating enhanced treatment planning ancillary data, by executing instructions or data from the image processing logic 120.

The image processing computing system 110 may include a processing circuit 112, a memory 114, a storage apparatus 116, and other hardware and software operational features such as a user interface 140 and a communication interface. The storage apparatus 116 may store computer-executable instructions for, for example, an operating system, a radiotherapy treatment plan (e.g., a raw treatment plan, or a modified treatment plan), a software program (e.g., radiotherapy treatment planning software and an artificial intelligence implementation such as a deep learning model, a machine learning model, or a neural network), and any other computer-executable instructions to be executed by the processing circuit 112.

In an example, the processing circuit 112 may include a processing apparatus, for example, one or more general-purpose processing apparatuses such as a microprocessor, a central processing unit (CPU), a graphics processing unit (GPU), or an accelerated processing unit (APU). More particularly, the processing circuit 112 may be a complex instruction set computing (CISC) microprocessor, a reduced instruction set computing (RISC) microprocessor, a very long instruction word (VLIW) microprocessor, a processor implementing another instruction set, or a processor implementing a combination of instruction sets. The processing circuit 112 may alternatively be implemented by one or more special-purpose processing apparatuses such as an application specific integrated circuit (ASIC), a field programmable gate array (FPGA), a digital signal processor (DSP), or a system on a chip (SoC). As will be understood by those skilled in the art, in some examples, the processing circuit 112 may be a special-purpose processor rather than a general-purpose processor. The processing circuit 112 may include one or more known processing apparatuses, for example, any processor from Pentium™, Core™, Xeon™, and Itanium® series microprocessors manufactured by Intel™, Turion™, Athlon™, Sempron™, Opteron™, FX™, and Phenom™ series microprocessors manufactured by AMD™, or various processors manufactured by Sun Microsystems. The processing circuit 112 may also include a graphics processing unit such as a GeForce®, Quadro®, or Tesla® series GPU manufactured by Nvidia™, a GMA or Iris™ series GPU manufactured by Intel™, or a Radeon™ series GPU manufactured by AMD™. The processing circuit 112 may further include, for example, a Xeon Phi™ series accelerated processing unit manufactured by Intel™. The disclosed implementation is not limited to any type of processor that is otherwise configured to meet a computational need of identifying, analyzing, maintaining, generating, and/or providing large amounts of data or manipulating such data to perform the method disclosed herein. Further, the term “processor” may include more than one processor, for example, a processor having a multi-core design or a plurality of processors each having a multi-core design. The processing circuit 112 may execute a sequence of computer program instructions stored in the memory 114 and accessed from the storage apparatus 116, to perform various operations, processing, and methods that will be described in more detail below.

The memory 114 may include a read-only memory (ROM), a phase-change random access memory (PRAM), a static random access memory (SRAM), a flash memory, a random access memory (RAM), a dynamic random access memory (DRAM) such as a synchronous DRAM (SDRAM), an electrically erasable programmable read-only memory (EEPROM), a static memory (e.g., a flash memory, a flash drive, or a static random access memory) and another type of random access memory, a cache memory, a register, a compact disc read-only memory (CD-ROM), a digital versatile disc (DVD) or another optical storage apparatus, a cassette tape, another magnetic storage apparatus, or any other non-transitory medium that may be used to store information including images, data, or computer-executable instructions (e.g., stored in any format) that can be accessed by the processing circuit 112 or any other type of computer apparatus. For example, the computer program instructions can be accessed by the processing circuit 112, may be read from the ROM or any other suitable memory location, and can be loaded into the RAM for execution by the processing circuit 112.

The storage apparatus 116 may constitute a drive unit that includes a machine-readable medium having stored thereon one or more instruction sets and data structures (e.g., software) (including, in various examples, the patient state processing logic 120 and the user interface 140) implemented or used by any one or more of the methods or functions described herein. During execution of instructions by the image processing computing system 110, the instructions may further reside in the memory 114 and/or in the processing circuit 112 in whole or at least in part, wherein the memory 114 and the processing circuit 112 also constitute a machine-readable medium.

The memory apparatus 114 and the storage apparatus 116 may constitute a non-transitory computer-readable medium. For example, the memory apparatus 114 or the storage apparatus 116 may store or load instructions for one or more software applications onto the computer-readable medium. Software applications stored or loaded with the memory apparatus 114 or the storage apparatus 116 may include, for example, an operating system for a general-purpose computer system and an operating system for an apparatus used for software control. The image processing computing system 110 may also operate various software programs including software code for implementing the image processing logic 120 and the user interface 140. Further, the memory apparatus 114 and the storage apparatus 116 can store or load an entire software application, a portion of a software application, or code or data associated with a software application that can be executed by the processing circuit 112. In another example, the memory apparatus 114 or the storage apparatus 116 may store, load, or manipulate one or more radiotherapy treatment plans, imaging data, patient state data, dictionary entries, artificial intelligence model data, labels, mapping data, and the like. It may be expected that software programs may be stored not only on the storage apparatus 116 and the memory 114, but also on a removable computer medium such as a hard disk drive, a computer disk, a CD-ROM, a DVD, an HD, a Blu-ray DVD, a USB flash drive, an SD card, a memory stick, or any other suitable medium. Such software programs may also be transmitted or received over a network.

Although not depicted, the image processing computing system 110 may include a communication interface, a network interface card, and a communication circuit. An exemplary communication interface may include, for example, a network adapter, a cable connector, a serial connector, a USB connector, a parallel connector, a high-speed data transmission adapter (e.g., an optical fiber, a USB 3.0, or a thunderbolt), a wireless network adapter (e.g., IEEE 802.11/Wi-Fi adapter), or a telecommunications adapter (e.g., communicating with 3G, 4G/LTE, and 5G networks). Such a communication interface may include one or more digital and/or analog communication apparatuses that allow a machine to communicate with another machine and apparatus, such as a remotely located part, via a network. The network can provide functions of a local area network (LAN), a wireless network, a cloud computing environment (e.g., software as a service, platform as a service, or infrastructure as a service.), a client-server, a wide area network (WAN), or the like. For example, the network may be a LAN or WAN that may include another system (including an additional image processing computing system or an image-based part associated with medical imaging or a radiotherapy operation).

In an example, the image processing computing system 110 may obtain image data 160 from the image data source 150, and the image data may be hosted on the storage apparatus 116 and the memory 114. In an example, a software program running on the image processing computing system 110 may, for example, convert a medical image in one format (e.g., MRI) into another format (e.g., CT) by generating a composite image such as a pseudo-CT image. In another example, the software program may register or associate a medical image (e.g., a CT image or an MR image) of a patient with dose distribution (e.g., also represented as an image) of a radiotherapy treatment for the patient, thereby allowing a corresponding image voxel to be appropriately associated with a dose voxel. In still another example, the software program may replace a function of a patient image, for example, a signed distance function or a processed version of an image that emphasizes some aspects of image information. Such a function may emphasize an edge or a difference or another structural aspect of a voxel texture. In another example, the software program can visualize, hide, emphasize, or de-emphasize certain aspects of anatomical features, patient measurements, patient state information, or dose or therapy information within a medical image. The storage apparatus 116 and the memory 114 may store and host data used to carry out these purposes, including the image data 160, patient data, and other data needed to create and implement a radiotherapy treatment plan and an associated patient state estimation operation.

The processing circuit 112 may be communicatively coupled to the memory 114 and the storage apparatus 116, and the processing circuit 112 may be configured to execute computer-executable instructions that are stored on the processing circuit and that come from the memory 114 or the storage apparatus 116. The processing circuit 112 may execute the instructions to cause a medical image from the image data 160 to be received or obtained in the memory 114 and processed using the patient state processing logic 120. For example, the image processing computing system 110 may receive the image data 160 from the image acquisition apparatus 170 or the image data source 150 via the communication interface and the network, so that the image data is stored or cached in the storage apparatus 116. The processing circuit 112 may further send or update the medical image stored in the memory 114 or the storage apparatus 116 to another database or data storage (e.g., a medical apparatus database) via the communication interface. In some examples, one or more systems may form a distributed computing/simulation environment that uses a network to cooperatively perform the implementations described herein. In addition, such a network may be connected to the Internet to communicate with a server and a client that reside remotely on the Internet.

In another example, the processing circuit 112 may use a software program (e.g., treatment planning software), the image data 160, and other patient data to create a radiotherapy treatment plan. In an example, the image data 160 may include, for example, 2D or 3D images from CT or MR. Further, the processing circuit 112 may use a deep neural network to process the image data 160, for example, may use the deep neural network to automatically obtain a region of interest in a tomographic image by segmentation.

The processing circuit 112 may further send an executable radiotherapy treatment plan to the treatment apparatus 180 via the communication interface and the network. In this treatment apparatus 180, the radiotherapy plan will be used to treat a patient with radiation via the treatment apparatus.

As discussed herein (e.g., with reference to enhanced image processing discussed herein), the processing circuit 112 may execute a software program that invokes the image processing logic 120 to implement functions including enhanced image generation.

In an example, the image data 160 may include one or more MRI images (e.g., 2D MRI, 3D MRI, 2D flow MRI, 4D MRI, 4D volumetric MRI, or 4D cine MRI images), functional MRI images (e.g., fMRI, DCE-MRI, or diffusion MRI images), computed tomography (CT) images (e.g., 2D CT, cone-beam CT, 3D CT, or 4D CT images), ultrasound images (e.g., 2D ultrasound, 3D ultrasound, or 4D ultrasound images), positron emission tomography (PET) images, X-ray images, fluoroscopic images, radiotherapy portal images, single photon emission computed tomography (SPECT) images, computer-generated composite images (e.g., pseudo-CT images), and the like. Further, the image data 160 may also include or be associated with medical image processing data such as a training image, a ground truth image, a contour image, and a dose image. In an example, the image data 160 may be received from the image acquisition apparatus 170 and the image data 160 is stored in one or more of the image data sources 150 (e.g., a picture archiving and communication system (PACS), a vendor neutral archive (VNA), a medical record or information system, or a data repository). Thus, the image acquisition apparatus 170 may include an MRI imaging apparatus, a CT imaging apparatus, a PET imaging apparatus, an ultrasound imaging apparatus, a fluoroscope apparatus, a SPECT imaging apparatus, an integrated linear accelerator and MRI imaging apparatus, or another medical imaging apparatus for obtaining a medical image of a patient. The image data 160 may be received and stored in any data type or any type of format (e.g., in a medical digital imaging and communication (DICOM) format) that the image acquisition apparatus 170 and the image processing computing system 110 may use to perform operations consistent with those in the disclosed implementations.

In an example, the image acquisition apparatus 170 may be integrated with the treatment apparatus 180 into a single device (e.g., an MRI apparatus combined with a linear accelerator, also referred to as an “MR linear accelerator”, as shown and described below in FIG. 3). Such an MRI linear accelerator may be used, for example, to accurately determine a position of a target organ or target tumor in a patient's body to accurately direct a radiotherapy to a predetermined target according to the radiotherapy treatment plan. For example, the radiotherapy treatment plan may provide information about a specific radiation dose to be applied to each patient. The radiotherapy treatment plan may also include other radiotherapy information such as a beam angle, dose-histogram-volume information, a quantity of radiation beams to be used during treatment, or dose per beam.

The image processing computing system 110 may communicate with an external database over a network to send/receive a plurality of various types of data related to image processing and radiotherapy operations. For example, the external database may include machine data. The machine data is information associated with the treatment apparatus 180, the image acquisition apparatus 170, or another machine related to a radiotherapy or a medical procedure. Machine data information may include a radiation beam size, arc placement, beam on and off duration, a machine parameter, a segment, a multi-leaf collimator (MLC) configuration, a gantry speed, an MRI pulse sequence, and the like. The external database may be a storage apparatus and may be provided with an appropriate database management software program. Further, such a database or data source may include a plurality of apparatuses or systems positioned in a centralized or distributed manner.

The image processing computing system 110 may collect and obtain data and communicate with another system via a network through one or more communication interfaces. The one or more communication interfaces may be communicatively coupled to the processing circuit 112 and the memory 114. For example, the communication interface may provide a communicative connection between the image processing computing system 110 and parts of a radiotherapy system (e.g., allowing data to be exchanged with an external apparatus). For example, in some examples, the communication interface may have an appropriate interface circuit with an output apparatus 142 or an input apparatus 144 to connect to the user interface 140, and the user interface 140 may be a hardware keyboard, keypad, or touch screen through which a user may input information into the radiotherapy system.

As an example, the output apparatus 142 may include a display apparatus. The display apparatus outputs a representation of the user interface 140 as well as one or more aspects, visualizations, or representations of medical images. The output apparatus 142 may include one or more display screens. The display screens display medical images, interface information, treatment plan parameters (e.g., a contour, a dose, a beam angle, a label, or a map), treatment plans, targets, target positioning or target tracking, patient state estimation (e.g., 3D images), or any information related to the user. The input apparatus 144 connected to the user interface 140 may be a keyboard, a keypad, a touch screen, or any type of apparatus through which a user may input information into the radiotherapy system. Alternatively, features of the output apparatus 142, the input apparatus 144, and the user interface 140 may be integrated into a single apparatus such as a smart phone or a tablet computer (e.g., Apple iPad®, LenovoThinkpad®, or Samsung Galaxy®).

Further, any and all parts of the radiotherapy system can be implemented as a virtual machine (e.g., via a virtualization platform of VMWare, Hyper-V, or the like). For example, the virtual machine may be software that functions as hardware. Thus, the virtual machine may include at least one or more virtual processors, one or more virtual memories, and one or more virtual communication interfaces that collectively function as hardware. For example, the image processing computing system 110, the image data source 150, or a similar part may be implemented as a virtual machine or implemented within a cloud-based virtualization environment.

The image processing logic 120 or another software program may cause the computing system to communicate with the image data source 150 to read an image into the memory 114 and the storage apparatus 116, or to store an image or associated data from the memory 114 or the storage apparatus 116 to the image data source 150 and store the image or associated data from the image data source 150 to the memory 114 or the storage apparatus 116. For example, the image data source 150 may be configured to store and provide a plurality of images (e.g., 3D MRI slice images, 4D MRI slice images, 2D MRI slice images, CT images, 2D fluoroscopy images, X-ray images, raw data from MR scanning or CT scanning, or medical digital imaging and communication (DICOM) metadata) that are hosted by the image data source 150 and that come from an image set in the image data 160 obtained from one or more patients by the image acquisition apparatus 170. The image data source 150 or another database may also store data to be used by the patient state processing logic 120 when a software program for performing a patient state estimation operation is executed or when a radiotherapy treatment plan is created. Further, various databases may store data generated by a preliminary motion model (e.g., a dictionary), a correspondence motion model, or a machine learning model, including network parameters and resulting prediction data that constitute a model learned through a network. In connection with performing image patient state estimation as part of a treatment or diagnostic operation, the image processing computing system 110 may thus obtain and/or receive the image data 160 (e.g., 2D MRI slice images, CT images, 2D fluoroscopy images, X-ray images, 3D MRI images, or 4D MRI images) from the image data source 150, the image acquisition apparatus 170, the treatment apparatus 180 (e.g., an MRI linear accelerator), or another information system.

The image acquisition apparatus 170 may be configured to acquire one or more images of a patient's anatomical structure for a region of interest (e.g., a target organ, a target tumor, or both). Each image, typically a 2D image or slice, may include one or more parameters (e.g., a thickness, an orientation, and a position of a 2D slice). In an example, the image acquisition apparatus 170 may obtain a 2D slice in any orientation. For example, the orientation of the 2D slice may include a sagittal orientation, a coronal orientation, or an axial orientation. The processing circuit 112 may adjust one or more parameters such as the thickness and/or orientation of the 2D slice to include a target organ and/or a target tumor. In an example, a 2D slice may be determined based on information such as a 3D MRI volume. When the patient is receiving radiotherapy treatment, for example, when the treatment apparatus 180 is used, such a 2D slice may be obtained “in real time” by the image acquisition apparatus 170 (“in real time” means obtaining data in 10 milliseconds or less). In another example for some applications, “in real time” may include a time range within (e.g., up to) 200 milliseconds or 300 milliseconds. In an example, “in real time” may include a time period that is fast enough to resolve a clinical problem with the technology described herein. In this example, “in real time” may be different depending on a target speed, a radiotherapy margin, a time lag, a response time of the treatment apparatus, or the like.

The image processing logic 120 in the image processing computing system 110 is depicted as processing a 4D data set of the patient to generate enhanced treatment planning ancillary data. In an example, the image processing logic 120 obtains a 4D data set of an object (e.g., a patient). The 4D data set contains a plurality of subsets of tomographic images of the object. The plurality of subsets respectively correspond to different positions in a scanning axial direction (direction z). The image processing logic 120 then segments the tomographic images in the subsets to separately obtain a region of interest (ROI) in each tomographic image. The image processing logic 120 further generates a region of interest contour based on the regions of interest. In another example, after obtaining the 4D data set of the object and obtaining, by segmenting the tomographic images, the region of interest in each tomographic image, the image processing logic 120 generates a probability distribution of pixels within a range of the region of interest contour of the corresponding subset. The region of interest contour and/or probability distribution may be used as enhanced treatment planning ancillary data and provided together with the 4D data set, to allow a physician to perform treatment planning (also referred to as making a treatment plan).

The treatment apparatus 180 may be a radiotherapy apparatus that includes, for example, an X-ray source or a radiation source of a linear accelerator, a treatment couch, an imaging detector, and a radiotherapy output portion. The treatment apparatus 180 may be configured to emit a radiation beam to provide treatment to a patient. The radiotherapy output portion may include one or more attenuators or collimators, such as multi-leaf collimators (MLCs). The patient may be positioned on and supported by the treatment couch to receive a radiotherapy dose according to a radiotherapy treatment plan (e.g., a treatment plan generated by the radiotherapy system of FIG. 1).

During treatment planning, a physician or technician may obtain image data, such as a 3D or 4D data set, of an object (e.g., a patient) in advance through the image acquisition apparatus 170. The 4D data set contains 3D data sets at different time points. The 3D data set contains a plurality of subsets of tomographic images. Each subset corresponds to one position in the scanning axial direction (z-axis in FIG. 1).

As an example, the image acquisition apparatus 170 may include an x-ray radiation source and a detector array. The x-ray radiation source may project a sector-shaped or cone-shaped x-ray beam. The x-ray beam penetrates the object, and enters the detector array after being attenuated by the object. The intensity of an attenuated radiation beam received at the detector array depends on the attenuation of the x-ray beam by the object. Each detector element of the detector array produces a separate electrical signal that serves as a measure of the intensity of the beam at a detector position. The image acquisition apparatus 170 may cause the x-ray radiation source and the detector array to rotate around the object in an imaging plane, so that an angle at which the x-ray beam intersects the object constantly changes. A “scan” of the object includes a set of views made at different gantry angles or viewing angles during one rotation of the x-ray radiation source 104 and the detector array 108. The image acquisition apparatus 170 may implement scanning of different axial positions by translating a workbench supporting the object in the scanning axial direction. As the object moves with the workbench, a plurality of sets of projection data may be obtained at certain time intervals to reconstruct a plurality of slice images. This set of slice images constitutes the 3D data set described herein. A plurality of slice images at the same scanning axial position may be acquired at different time points, to obtain a 4D data set. The plurality of slice images at the same scanning axial position constitute a subset. If a tomographic portion of the object corresponding to the scanning axial position contains a movable portion, the subset can reflect a motion status of the movable portion. In some embodiments, a scanning time period for the same subset may exceed one respiratory cycle of the object. The respiratory cycle of the object may be predetermined. For example, the respiratory cycle of the object may be obtained through the D4D or RGSC method described above.

The 4D data set of the object is useful to a physician. During treatment planning, for example, dose planning, the physician may estimate a motion status of a region of interest (e.g., a tumor or a vital organ) based on the 4D data set to perform the dose planning. Typically, the physician performs the dose planning by observing images and relying on experience. A region involved in the dose planning is usually larger than an actual region of interest in a single tomographic image, to ensure that a radiation dose covers all regions of interest. However, an empirically estimated radiation dose may cause an excessive radiation dose to a normal portion (tissue) surrounding the regions of interest, resulting in unnecessary negative effects.

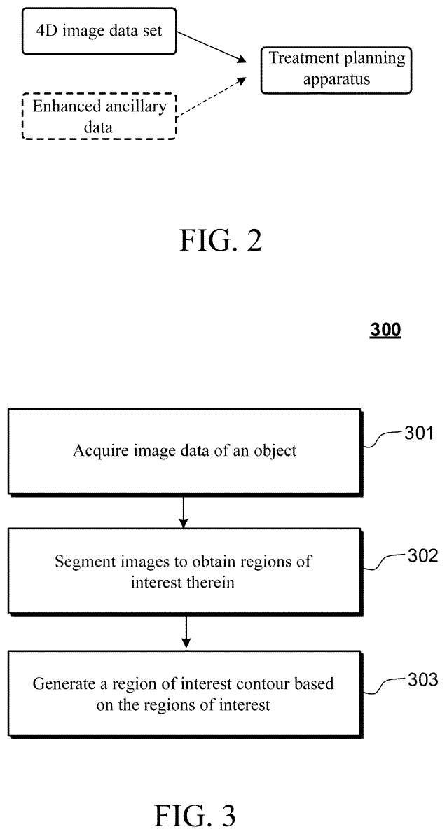

The present disclosure utilizes enhanced ancillary data to assist the physician in performing treatment planning, and in particular in performing more accurate dose planning. FIG. 2 illustrates a schematic diagram of treatment planning incorporating enhanced ancillary data according to an embodiment of the present disclosure.

A treatment planning apparatus may be the computing system 110 described with reference to FIG. 1, in which the treatment planning software, the image data 160, and other patient data are deployed to create a radiotherapy treatment plan. The image data 160 is a 4D image data set obtained through the image acquisition apparatus 170.

The image processing logic 120 may generate enhanced ancillary data based on the 4D image data set. The enhanced ancillary data may be provided to the treatment planning apparatus together with a raw 4D image data set, rather than merely providing the 4D image data set.

The enhanced ancillary data contains motion information of a region of interest in the raw 4D image data set. The motion information may include a range and frequency of occurrence of the region of interest. The range of the region of interest may be used to determine a range of dose planning. The frequency of occurrence may be used to determine a planned dose at each position.

FIG. 3 illustrates a flowchart of an exemplary method 300 of generating enhanced ancillary data according to an embodiment of the present disclosure. In block 301, image data of an object (e.g., a patient) is obtained. The image data may be the 4D data set described above, which is obtained by scanning the object by the image acquisition apparatus 170 shown in FIG. 1, and includes a plurality of subsets of tomographic images of the object. The plurality of subsets respectively correspond to different positions in a scanning axial direction (z-axis in FIG. 1, along which the object is arranged on a support platform). Each subset includes a plurality of tomographic images, at different time points of a specific scanning time period, obtained by scanning the object at a specific position in the scanning axial direction. Some or all of the tomographic images may contain a movable portion of the object. Thus, each subset can reflect a motion status of the movable portion at the specific position in the scanning time period. The scanning time period, as described above, may be a time period greater than one respiratory cycle of the object. The respiratory cycle may be predetermined through the D4D, the RGSC, or another method described herein.

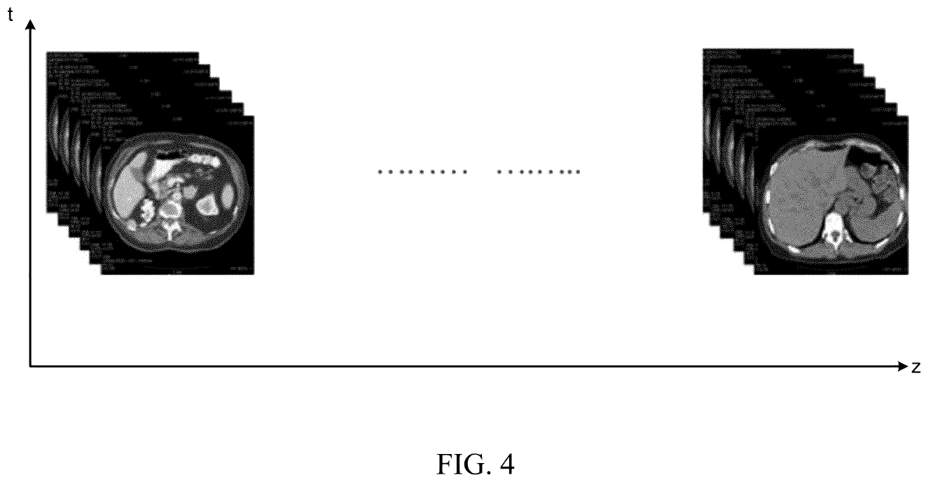

FIG. 4 illustrates a schematic diagram of a 4D data set, wherein the abscissa represents a position in the scanning axial direction (z-axis) and the ordinate represents time. In the figure, a set of overlapping tomographic images represents a subset, corresponding to the same axial scanning position. Different scanning time periods and/or scanning intervals may be set between different subsets. In other words, a specific scanning time period of at least one of the plurality of subsets may be different from those of the other subsets.

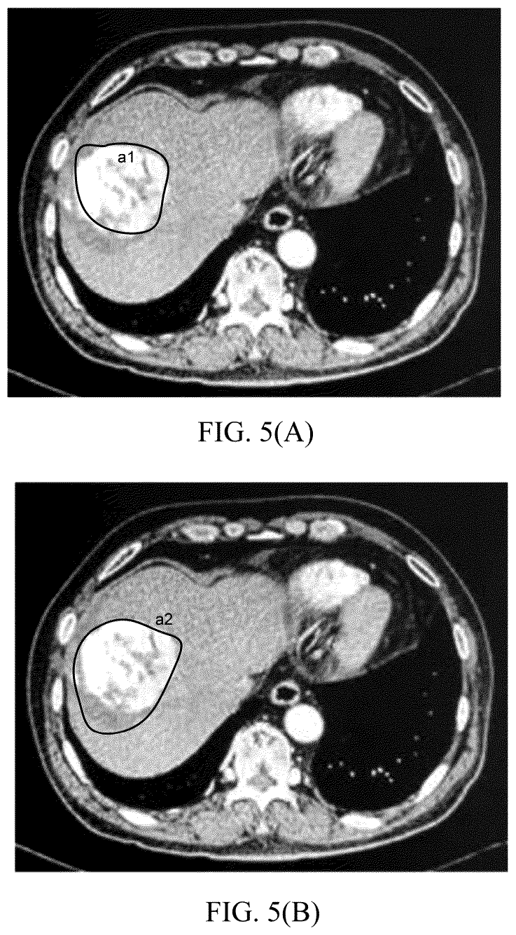

Next, in block 302, each tomographic image of one or a plurality of subsets is segmented to separately obtain a region of interest in each tomographic image. FIG. 5(A) to FIG. 5(C) illustrate exemplary tomographic images that are taken at different time points at the same scanning axial position and may be tomographic images in a certain subset in FIG. 4. In FIG. 5(A) to FIG. 5(C), portions surrounded by solid lines represent regions of interest obtained by segmentation. The region of interest may be a tumor, a critical organ, or the like.

In some implementations, the region of interest may be obtained by automatic segmentation via deep learning. To improve segmentation accuracy, a user may define, via a user input, a position range in which the region of interest is located. For example, the user may mark an approximate range of the region of interest in a certain tomographic image in the subset. The marking of the approximate range can be implemented through a mouse, a touch operation, or the like. A specific position or a specific region may be selected to mark the approximate range. Alternatively, the user may select, through a list, a menu, or the like, a type (e.g., a tumor or a critical organ) of a region of interest that needs to be obtained by segmentation. Based on the user input, the image processing logic 120 may perform segmentation for regions of interest in all tomographic images in a corresponding subset, to obtain results shown in FIG. 5(A) to FIG. 5(C), as shown by a1 to a3 in the figures.

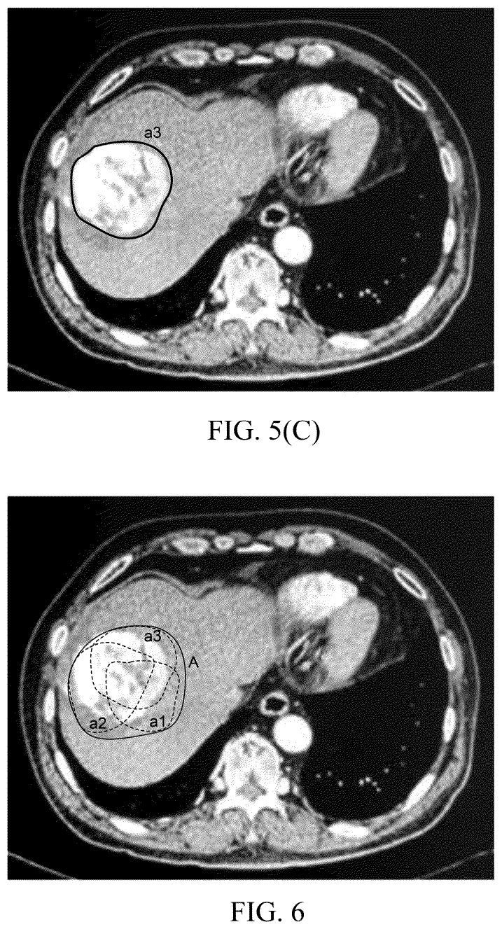

Next, in block 303, the image processing logic 120 generates a region of interest contour of the subset based on regions of interest obtained by segmentation of the plurality of tomographic images in the subset. FIG. 6 illustrates a schematic diagram of an exemplary region of interest contour according to an embodiment of the present disclosure. The image processing logic 120 generates a region of interest contour A (as shown by a solid line) of the current subset based on the regions of interest (a1 to a3 shown by dashed lines) obtained by segmentation (through the block 302) of all tomographic images in the current subset.

The region of interest contour A is generated by superimposing contours of the regions of interest a1 to a3 of all the tomographic images in the same subset, such that pixels within the regions of interest a1 to a3 of all the tomographic images in the same subset are located within the region of interest contour A, and any pixel within the region of interest contour A is located within the regions of interest a1 to a3 of at least one of all the tomographic images of the corresponding subset.

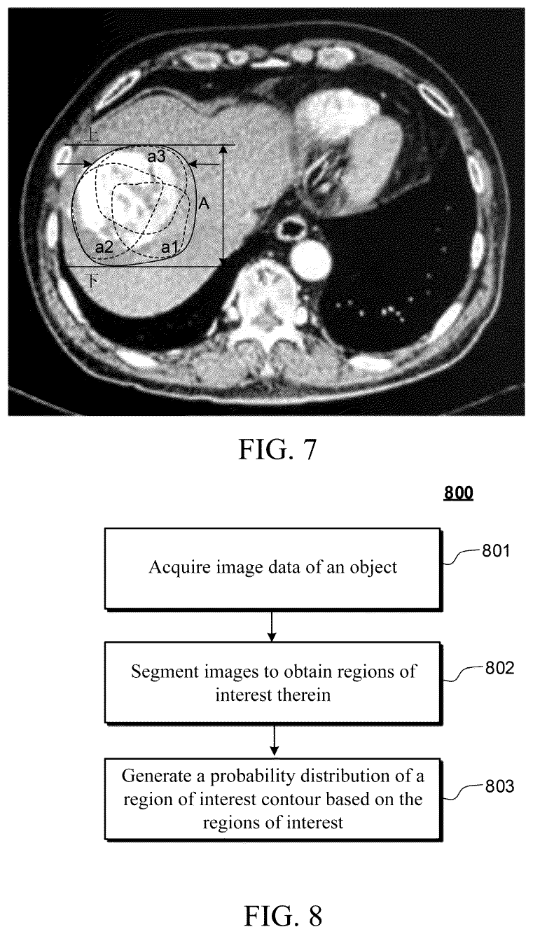

FIG. 7 illustrates an exemplary generation method of a region of interest contour according to an embodiment of the present disclosure. In this embodiment, the region of interest contour may be determined row by row, for example, in a top-down order. The contour of each row may be determined based on the outermost boundary of a plurality of regions of interest of the same row. Specifically, for a row, the left and right coordinates of the region of interest contour need to be determined. The left coordinate may be determined as a coordinate of a boundary point located on the leftmost side of the corresponding row in all the regions of interest. Similarly, the right coordinate may be determined as a coordinate of a boundary point located on the rightmost side of the corresponding row in all the regions of interest. The foregoing process of determining left and right coordinates is performed for each row in which at least one region of interest segment exists. Therefore, the region of interest contour may be determined based on the left and right coordinates of all rows in which at least one region of interest segment exists.

It will be understood that the region of interest contour is determined based on a set of regions of interest in one subset of tomographic images, and represents a motion status of the regions of interest in the subset. The region of interest contour of each subset may be determined separately, to obtain a 3D data set of region of interest contours. The region of interest contour can reflect a motion range of the regions of interest in the subset, and thus can serve as enhanced treatment planning ancillary data.

After the region of interest contour is generated, the region of interest contour of each subset of tomographic images may be output to the treatment planning apparatus together with the raw 4D data set. In some implementations, the region of interest contour may be visually presented. In some implementations, a region of interest contour corresponding to a subset associated with at least one tomographic image may be displayed on the tomographic image. The region of interest contour may be displayed with transparency, so that a physician can simultaneously view the raw tomographic images and a range of the region of interest contour. The physician may perform treatment planning, for example, determine an irradiation range, based on the region of interest contour of each subset, that is, the 3D data set of the region of interest contours.

The region of interest contour determined in the manner according to this embodiment can more accurately describe the motion range of the regions of interest, reducing the possibility of incorporating an unnecessary healthy tissue into the region of interest contour. Therefore, performing treatment planning based on the region of interest contour determined in this embodiment can significantly reduce an unnecessary radiation dose to healthy tissues.

FIG. 8 illustrates a flowchart of another exemplary method 800 of generating enhanced ancillary data according to an embodiment of the present disclosure. In block 801, image data of an object (e.g., a patient) is obtained. The image data may be the 4D data set described above, which is obtained by scanning the object by the image acquisition apparatus 170 shown in FIG. 1, and includes a plurality of subsets of tomographic images of the object. The plurality of subsets respectively correspond to different positions in a scanning axial direction (z-axis in FIG. 1, along which the object is arranged on a support platform). Each subset includes a plurality of tomographic images, at different time points of a specific scanning time period, obtained by scanning the object at a specific position in the scanning axial direction. Some or all of the tomographic images may contain a movable portion of the object. Thus, each subset can reflect a motion status of the movable portion at the specific position in the scanning time period. The scanning time period, as described above, may be a time period greater than one respiratory cycle of the object. The respiratory cycle may be predetermined through the D4D, the RGSC, or another method described herein.

Next, in block 802, each tomographic image in one or a plurality of subsets is segmented to separately obtain a region of interest in each tomographic image, for example, as shown in FIG. 5(A) to FIG. 5(C). The region of interest may be obtained by automatic segmentation via deep learning. A user may define, via a user input, a position range in which the region of interest is located. For example, the user may mark an approximate range of the region of interest in a certain tomographic image in the subset. The marking of the approximate range can be implemented through a mouse, a touch operation, or the like. A specific position or a specific region may be selected to mark the approximate range. Alternatively, the user may select, through a list, a menu, or the like, a region of interest that needs to be obtained by segmentation. Based on the user input, the image processing logic 120 may perform segmentation for regions of interest in all tomographic images in a corresponding subset.

Next, in block 803, the image processing logic 120 generates a probability distribution of pixels within a range of a region of interest contour of the subset based on regions of interest obtained by segmentation of the plurality of tomographic images in the subset. The probability distribution described herein is a set of probabilities that respective pixels in the region of interest contour belong to regions of interest. The region of interest contour may be determined, as described above, based on the regions of interest of all the tomographic images in the corresponding subset.

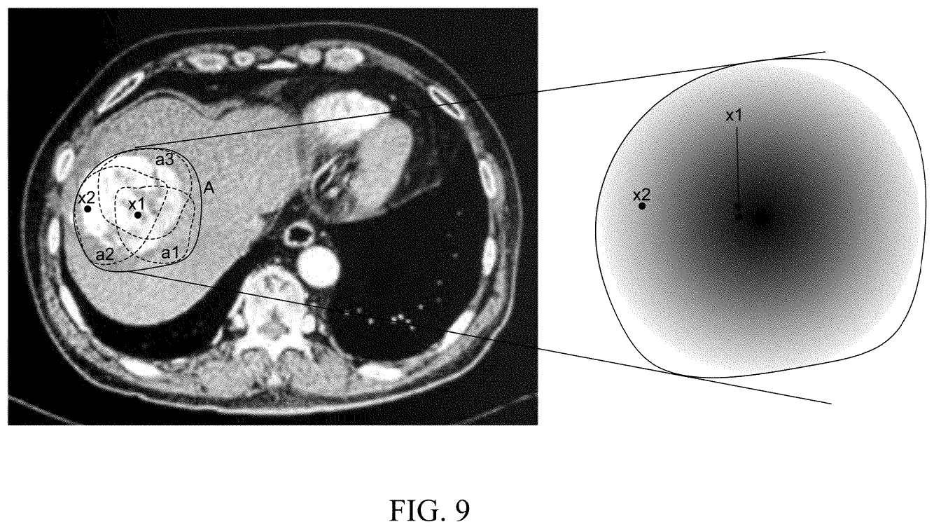

FIG. 9 illustrates a schematic diagram of an exemplary probability distribution according to an embodiment of the present disclosure. The region of interest contour described herein is formed by superimposing the regions of interest in the same subset of tomographic images. However, the probability that each pixel in the region of interest contour becomes a region of interest is different. For example, in an example of FIG. 9, a point x1 appears in all of regions of interest a1, a2, a3, while a point x2 appears only in the region of interest a2. Therefore, although both the point x1 and the point x2 are located within the region of interest contour A, the probability that both belong to a region of interest is different. For example, the probability of the point x1 is 100%, and the probability of the point x2 is 33.33%.

In block 803, the probability that each pixel in the region of interest contour belongs to a region of interest is calculated. Specifically, the probability that each pixel belongs to a region of interest is calculated through Formula (1):

$${P}_{Z , x , y} = \frac{{Appearance}_{Z , x , y}}{CurtMaxAppearance} \qquad ( 1 )$$

Z represents a coordinate in the scanning axial direction (direction z), x and y respectively represent an abscissa and an ordinate in a tomographic image plane perpendicular to the scanning axial direction, Appearance_{Z,x,y} represents a quantity of times a current pixel is covered by regions of interest of all tomographic images in a corresponding subset, CurtMaxAppearance represents the maximum Appearance_{Z,x,y} of the pixel in the current subset, and the maximum Appearance_{Z,x,y} does not exceed a total quantity of regions of interest obtained by segmentation in the current subset. The probability distribution of the region of interest contour can be obtained by calculating a probability value of each pixel in the region of interest contour. The probability distribution helps a physician determine a specific dose distribution when the physician performs treatment planning.

It should be understood that in block 803, the region of interest contour may not be generated according to the method described with reference to block 303. It may be determined for each pixel whether it is located in at least one region of interest obtained by segmentation in the plurality of tomographic images of the subset. If the pixel is not in any region of interest, the probability that the pixel belongs to a region of interest may be determined to be 0. If the pixel is located in at least one region of interest, the probability can be calculated through Formula (1). Finally, a contour of a set of all pixels with probabilities greater than 0 is equivalent to the region of interest contour.

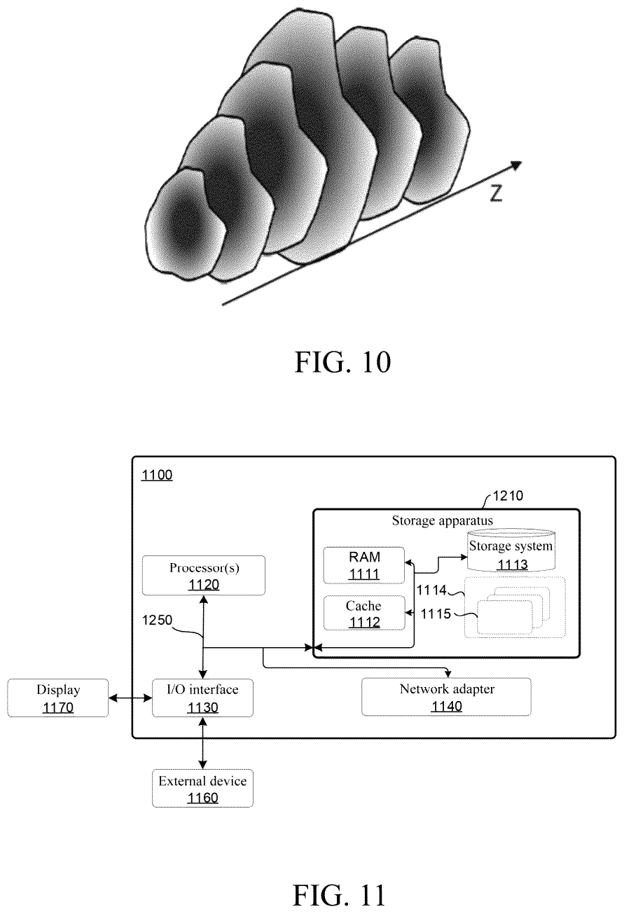

FIG. 10 illustrates a schematic diagram of an exemplary probability distribution of a plurality of subsets according to an embodiment of the present disclosure. The probability distribution calculated for subsets at different positions along the z-axis (scanning axial direction) is shown in the figure. It should be noted that, as described above, a different scanning time period and sampling interval may be separately set for each subset. Thus, for each subset, a base, that is, CurtMaxAppearance, for calculating the probability distribution may be different, and because quantities of tomographic images contained in different subsets may be different, the total quantities of regions of interest obtained by segmentation may also be different. As a result, the maximum Appearance_{Z,x,y} of the pixel in each subset may also be different. To normalize the probability distribution calculated for each subset, the probability may be calculated using all subsets as a base, that is, the probability distribution for each subset is calculated through the following Formula (2):

$${P}_{Z , x , y} = \frac{{Appearance}_{Z , x , y}}{MaxAppearance} \qquad ( 2 )$$

Z represents a coordinate in the scanning axial direction (direction z), x and y respectively represent an abscissa and an ordinate in a tomographic image plane perpendicular to the scanning axial direction, Appearance_{Z,x,y} represents a quantity of times a current pixel is covered by regions of interest of all tomographic images in a corresponding subset, and MaxAppearance represents the maximum Appearance_{Z,x,y} of pixels in all subsets of the 4D data set. Therefore, the probability distribution of each subset is calculated based on a pixel having the largest quantity of times of being covered by the regions of interest in all subsets, so that a set of normalized probability distributions can be obtained.

When the probability distribution is calculated by using Formula (2), a quantity of times each pixel of the 4D data set is covered by all regions of interest obtained by segmentation in a subset corresponding to the pixel may be determined, and the maximum quantity of times of being covered is used as MaxAppearance. The probability of each pixel is then calculated based on MaxAppearance.

In some implementations, the probability distribution calculated through the block 803 may be output to the treatment planning apparatus together with the raw 4D data set. The probability distribution may be visually presented. In some implementations, the probability distribution corresponding to a subset associated with at least one tomographic image may be displayed on the tomographic image. The probability distribution may be displayed with transparency, so that a physician can simultaneously view the raw tomographic images and a range of the probability distribution. Preferably, each pixel in the probability distribution may be displayed in a manner corresponding to the probability of the pixel, for example, displayed with a gray value. For example, a pixel having a higher probability of belonging to a region of interest may be displayed with a larger gray value. Therefore, the physician can intuitively grasp a motion status of the regions of interest to perform dose planning for each position.

The treatment planning apparatus described herein may be implemented by the computing system 110 described with reference to FIG. 1. The treatment planning apparatus may include an image acquisition unit not shown in a figure, configured to acquire a raw tomographic image data set of the object from the memory apparatus 114 and/or the storage apparatus 116 and obtain, from the image processing logic 120, enhanced treatment planning ancillary data generated in the manner described herein, for example, the region of interest contour and/or the probability distribution described herein. The processing circuit 112 may, via the user interface 140, cause the output apparatus 142 to present raw tomographic image data and the enhanced treatment planning ancillary data. As described above, the output apparatus 142 may present the region of interest contour in a manner corresponding to the enhanced treatment planning ancillary data, for example, in a manner of having transparency, and may also present the probability distribution in a manner corresponding to the probability of each pixel.

The processing circuit 112 may further receive an input from the input apparatus 144 via the user interface 140 to update a treatment plan in the storage apparatus 116 and then output an updated treatment plan to the treatment apparatus 180.

FIG. 11 illustrates an exemplary block diagram of a computing device 1100 according to a technology of the present disclosure. The computing device 1100 may be implemented as an example of the computing system 110 shown in FIG. 1. The computing device 1100 includes: one or a plurality of processors 1120; and a storage apparatus 1110, configured to store one or a plurality of programs, wherein when the one or plurality of programs are executed by the one or plurality of processors 1120, the one or plurality of processors 1120 are caused to implement the processes described in the present disclosure. The processor is, for example, a digital signal processor (DSP), a microcontroller, an application-specific integrated circuit (ASIC), or a microprocessor.

The computing device 1100 shown in FIG. 11 is merely an example, and should not impose any limitation to the function and usage scope of the embodiments of the present invention.

As shown in FIG. 11, the computing device 1100 is embodied in the form of a general-purpose computing device. Components of the computing device 1100 may include, but are not limited to: one or a plurality of processors 1120, a storage apparatus 1110, and a bus 1150 connecting different system components (including the storage apparatus 1110 and the processor 1120).

The bus 1150 represents one or a plurality of types among several types of bus structures, including a memory bus or a memory controller, a peripheral bus, a graphics acceleration port, a processor, or a local bus using any of the plurality of bus structures. For example, these architectures include, but are not limited to, an industry standard architecture (ISA) bus, a microchannel architecture (MAC) bus, an enhanced ISA bus, a video electronics standards association (VESA) local bus, and a peripheral component interconnection (PCI) bus.

The computing device 1100 typically includes a plurality of computer system-readable media. These media may be any available medium that can be accessed by the computing device 1100, including volatile and non-volatile media as well as movable and non-movable media.

The storage apparatus 1110 may include a computer system-readable medium in the form of a volatile memory, for example, a random access memory (RAM) 1111 and/or a cache memory 1112. The computing device 1100 may further include other movable/non-movable, and volatile/non-volatile computer system storage media. For example only, the storage system 1113 may be configured to read and write a non-removable non-volatile magnetic medium (which is not shown in FIG. 11, and is generally referred to as a “hard drive”). Although not shown in FIG. 11, a magnetic disk drive for reading and writing a removable non-volatile magnetic disk (such as a “floppy disk”) and an optical disc drive for reading and writing a removable non-volatile optical disc (such as a CD-ROM, a DVD-ROM, or other optical media) may be provided. In these cases, each drive may be connected to the bus 1150 through one or a plurality of data medium interfaces. The storage apparatus 1110 may include at least one program product, the program product has a group (for example, at least one) of program modules, and these program modules are configured to perform the functions of the various embodiments in the present invention.

A program/utility tool 1114 having a group (at least one) of program modules 1115 may be stored in, for example, the storage apparatus 1110. This program module 1115 includes, but is not limited to, an operating system, one or a plurality of application programs, other program modules, and program data, and each of these examples or a certain combination thereof may include an implementation of a network environment. The program module 1115 usually performs the function and/or method in any embodiment described in the present invention.

The computing device 1100 may also communicate with one or a plurality of peripheral devices 1160 (such as a keyboard, a pointing device, and a display 1170), and may also communicate with one or a plurality of devices that enable a user to interact with the computing device 1100, and/or communicate with any device (such as a network card and a modem) that enables the computing device 1100 to communicate with one or a plurality of other computing devices. Such communication may be performed via an input/output (I/O) interface 1130. Moreover, the computing device 1100 may also communicate with one or a plurality of networks (for example, a local area network (LAN), a wide area network (WAN) and/or a public network, for example, the Internet) through a network adapter 1140. As shown in FIG. 11, the network adapter 1140 communicates with other modules of the computing device 1100 through the bus 1150. It should be understood that although not shown in the figure, other hardware and/or software modules can be used in combination with the computing device 1100, including, but not limited to: microcode, device drivers, redundant processing units, external disk drive arrays, RAID systems, tape drives, data backup storage systems, and the like.

The processor 1120 executes various functional applications and data processing, for example implementing the processes described in the present disclosure, by running programs stored in the storage apparatus 1110.

The technique described herein may be implemented with hardware, software, firmware, or any combination thereof, unless specifically described as being implemented in a specific manner. Any features described as modules or parts may also be implemented together in an integrated logical device, or separately implemented as discrete but interoperable logical devices. If implemented with software, the technique may be implemented at least in part by a non-transitory processor-readable storage medium that includes instructions, wherein when executed, the instructions perform one or more of the aforementioned methods. The non-transitory processor-readable data storage medium may form part of a computer program product that may include an encapsulation material. Program code may be implemented in a high-level procedural programming language or an object-oriented programming language so as to communicate with a processing system. If desired, the program code may also be implemented in an assembly language or a machine language. In fact, the mechanisms described herein are not limited to the scope of any particular programming language. In any case, the language may be a compiled language or an interpreted language.

One or a plurality of aspects of at least some embodiments may be implemented by representative instructions that are stored in a machine-readable medium and represent various logic in a processor, wherein when read by a machine, the representative instructions cause the machine to manufacture the logic for executing the technique described herein.

Such machine-readable storage media may include, but are not limited to, a non-transitory tangible arrangement of an article manufactured or formed by a machine or device, including storage media, such as: a hard disk; any other types of disk, including a floppy disk, an optical disk, a compact disk read-only memory (CD-ROM), compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions.

Instructions may further be sent or received by means of a network interface device that uses any of a number of transport protocols (for example, Frame Relay, Internet Protocol (IP), Transfer Control Protocol (TCP), User Datagram Protocol (UDP), and Hypertext Transfer Protocol (HTTP)) and through a communication network using a transmission medium.

An exemplary communication network may include a local area network (LAN), a wide area network (WAN), a packet data network (for example, the Internet), a mobile phone network (for example, a cellular network), a plain old telephone service (POTS) network, and a wireless data network (for example, Institute of Electrical and Electronics Engineers (IEEE) 802.11 series standards referred to as Wi-Fi®, and IEEE 802.19 series standards referred to as WiMax®), IEEE 802.15.4 series standards, a peer-to-peer (P2P) network, and the like. In an example, the network interface device may include one or a plurality of physical jacks (for example, Ethernet, coaxial, or phone jacks) or one or a plurality of antennas for connection to the communication network. In an example, the network interface device may include a plurality of antennas that wirelessly communicate using at least one technique of single-input multiple-output (SIMO), multiple-input multiple-output (MIMO), or multiple-input single-output (MISO) techniques.

The term “transmission medium” should be considered to include any intangible medium capable of storing, encoding, or carrying instructions for execution by a machine, and the “transmission medium” includes digital or analog communication signals or any other intangible medium for facilitating communication of such software.

So far, the method and the apparatus for generating enhanced treatment planning ancillary data according to the present invention have been described, and a treatment planning system and a computer-readable storage medium capable of implementing the method have also been described.

According to the technology of the present disclosure, enhanced treatment planning ancillary data can be generated based on raw tomographic data of an object. The enhanced treatment planning ancillary data is a description of a motion range of regions of interest and thus can help a physician in performing more accurate treatment planning to reduce unnecessary radiation doses. Further, by presenting the enhanced treatment planning ancillary data together with the raw tomographic data, the physician can intuitively grasp a motion status of the regions of interest to perform treatment planning.

Some exemplary embodiments have been described above. However, it should be understood that various modifications can be made to the exemplary embodiments described above without departing from the spirit and scope of the present invention. For example, an appropriate result can be achieved if the described techniques are performed in a different order and/or if the components of the described system, architecture, device, or circuit are combined in other manners and/or replaced or supplemented with additional components or equivalents thereof; accordingly, the modified other implementations also fall within the protection scope of the claims.

1. A computer implemented method, comprising:
acquiring a plurality of subsets of tomographic images of an object, wherein the plurality of subsets respectively correspond to different positions in a scanning axial direction, and wherein each of the plurality of subsets comprises a plurality of tomographic images, at different time points of a specific scanning time period, obtained by scanning a movable portion of the object at a specific position in the scanning axial direction;
segmenting each of the plurality of tomographic images in one of the plurality of subsets to separately obtain a region of interest in each tomographic image; and
generating a region of interest contour of the one subset based on the regions of interest of the plurality of tomographic images in the one subset.

2. The method according to claim 1, further comprising: generating a probability distribution of pixels within a range of the region of interest contour of the one subset, wherein the probability distribution is a set of probabilities that respective pixels in the region of interest contour belong to regions of interest.

3. The method according to claim 2, wherein the probability distribution of the region of interest contour of the one subset is calculated using the one subset as a base.

4. The method according to claim 2, wherein the probability distribution of the region of interest contour of the one subset is calculated using the plurality of subsets as a base.

5. The method according to claim 1, wherein the region of interest contour is generated by superimposing contours of the regions of interest of all tomographic images in the same subset, such that pixels within the regions of interest of all the tomographic images in the same subset are located within the region of interest contour, and any pixel within the region of interest contour is located within the region of interest of at least one of all the tomographic images.

6. The method according to claim 1, wherein the region of interest is obtained by automatic segmentation via deep learning.

7. The method according to claim 1, further comprising: defining, via a user input, a position range in which the region of interest is located.

8. The method according to claim 1, wherein the specific scanning time period of each of the subsets exceeds one respiratory cycle of the object.

9. The method according to claim 1, wherein the specific scanning time period of at least one of the plurality of subsets is different from those of the other subsets.

10. The method according to claim 2, further comprising: outputting the region of interest contour and the probability distribution together with the one subset to a treatment planning apparatus.

11. The method according to claim 10, further comprising: displaying the region of interest contour in the tomographic images of the one subset, wherein the region of interest contour has transparency.

12. The method according to claim 10, wherein the region of interest contour of the one subset is presented in a manner corresponding to the probability distribution.

13. A computer implemented method, comprising:
acquiring a plurality of subsets of tomographic images of an object, wherein the plurality of subsets respectively correspond to different positions in a scanning axial direction, and wherein each of the plurality of subsets comprises a plurality of tomographic images, at different time points of a specific scanning time period, obtained by scanning a movable portion of the object at a specific position in the scanning axial direction;
segmenting each of the plurality of tomographic images in one of the plurality of subsets to separately obtain a region of interest in each tomographic image; and
generating a probability distribution of pixels within a range of a region of interest contour of the one subset, wherein the probability distribution is a set of probabilities that respective pixels in the region of interest contour belong to regions of interest, and the region of interest contour is determined based on the regions of interest of all tomographic images in the one subset.

14. The method according to claim 13, further comprising: displaying the region of interest contour in the tomographic images of the one subset, wherein the region of interest contour has transparency.

15. The method according to claim 13, wherein the region of interest contour of the one subset is presented in a manner corresponding to the probability distribution.

16. A treatment planning apparatus, comprising:
an image acquisition unit, configured to acquire a plurality of subsets of tomographic images of an object and a region of interest contour corresponding to one of the plurality of subsets, wherein the region of interest contour is generated by superimposing contours of regions of interest obtained by segmentation of all tomographic images in the one subset, such that pixels within the regions of interest of all the tomographic images in the one subset are located within the region of interest contour, and any pixel within the region of interest contour is located within the region of interest of at least one of all the tomographic images; and
a display, configured to present the region of interest contour corresponding to the one subset.

17. The treatment planning apparatus according to claim 16, wherein the display is further configured to: display the region of interest contour in the tomographic images of the one subset, and the region of interest contour has transparency.

18. The treatment planning apparatus according to claim 16, wherein
the image acquisition unit is further configured to acquire a probability distribution of the region of interest contour, wherein the probability distribution is a set of probabilities that respective pixels in the region of interest contour belong to regions of interest; and
the display is further configured to present the probability distribution of the region of interest contour corresponding to the one subset.

19. The treatment planning apparatus according to claim 18, wherein the display is further configured to: present the region of interest contour of the one subset in a manner corresponding to the probability distribution.
